(19) United States
(12) Patent Application Publication
(10) Pub. No.: US 2004/0022733 A1
(43) Pub. Date: Feb. 5, 2004

(54) Conjugated lysine copolymers

(76) Inventor: Egidijus E. Uzgiris, Niskayuna, NY (US)

Correspondence Address: Raymond E. Farrell, Esq., Carter, DeLuca. Farrell & Schmidt, LLP, Suite 225, 445 Broad Hollow Road, Melville, NY, 11747, US

(21) Appl. No.: 10/209,993

(22) Filed: Jul. 31, 2002

(51) Int. Cl. A61K 49/00; C07K 14/00

(52) U.S. Cl. 424/9.34; 530/400

(57) ABSTRACT

A biocompatible molecule includes a polypeptide containing lysine residues and either gluatmic acid or aspartic acid residues, less than 90% of the lysine residues being substituted with a group derived from a steric hindrance molecule, the substituted polypeptide having a conformation with a length that is 5 to 500 times its average diameter.

### BACKGROUND

1. Technical Field

The present disclosure relates to improved conjugated polymers for medical treatment of tumor tissue, and more specifically, for optimizing drug delivery to tumor tissue as well as to the diagnostic imaging of tumors.

2. Description of Related Art

In many medical procedures it is important to accumulate a certain active agent to a desired tissue type. For example, in chemotherapy, it is important to deliver drugs only to cancerous tumor tissue, and not to normal tissue, since these drugs destroy the tissue with which they come in contact. Another example would be in medical imaging. Contrast agents are attached to carrier molecules which are specific to tumor tissue. As the carrier molecules concentrate in the tumor tissue, the contrast agents enhance a medical image of this tissue.

The use of a chemotherapy drug (e.g., Doxorubicin) attached to Poly-L-Aspartic Acid (PAA) has been previously described. Many of the carrier molecules employed are proteins having a globular or folded configuration.

One known type of carrier molecule contains polypeptides having a diameter larger than pores of blood vessels of normal tissue and smaller than pores of blood vessels of tumor tissue. See, U.S. Pat. No. 5,762,909. These carriers have a length several orders of magnitude greater than their diameter, a net negative charge, and form a worm-like chain conformation with a long persistence length. Lanthanide complexes (e.g., gadolinium-diethylenetriamine pentaacetic acid complexes) are attached to these carrier molecules to create complex molecules which are introduced into a blood vessel of the subject.

These complex molecules pass though the pores of only the tumor endothelium and interact with the fibrous structures of the tumor interstitium. The penetration of the tumor interstitium by the complex molecules is enhanced by the worm-like configuration of the complex molecule which allows the molecule to “snake” around fixed obstacles in the extracellular matrix of the tumor interstitium.

The worm-like configuration of the complex molecule is achieved by attaching a sufficient number of diethylenetriamine pentaacetic acid (DTPA) molecules along the polypeptide chain to eliminate or reduce intra-chain ionic bonds as well to allow charge repulsion between DTPA moieties to unfold and extend the polymer chain. The amount of substitutions (also referred to as the degree of conjugation) thus affects the configuration of the resulting complex, with a higher degree of conjugation providing a more consistent extended structure and better targeting. Unfortunately, it is difficult to reliably attain degrees of conjugation of higher than 90%. (See, Sieving et al., Bioconjugate Chem., 1, 65 (1990).) Substitutions of above 90% are as rare as 1 in 7 synthesis runs, even with high anhydride to lysine ratios and extended reaction times. Yet, this level of substitution is required for the proper polymer configuration to be realized in the homopolymer case.

Accordingly, it would be advantageous to provide polypeptide-DTPA molecules that provide the desired conformation without requiring an extremely high degree of conjugation.

### SUMMARY

Substituted random copolymers of lysine acid and either glutamic or aspartic acid are employed to provide a backbone for carrier molecules having a diameter larger than the pores in blood vessels of normal tissue and smaller than pores of blood vessels of tumor tissue. The carrier molecule has a length in the range of 5 to 500 times greater than its diameter, and, preferably, a net negative charge. Surprisingly, the present carrier molecules form a worm-like chain conformation with a long persistence length despite the fact that the lysine residues possess a degree of conjugation of less than 90%. It is believed that in an aqueous environment the negatively charged glutamic acid groups contribute to the charge repulsion interactions that extend the chain. On the other hand, free lysines that are positively charged in an aqueous environment and contribute to polymer folding are fractionally reduced in the entire polymer chain. Thus, if for example, the ratio of glutamic acid to lysine in the polymer is 1:1, then 20% of unconjugated free lysines would represent only 10% of charges that are positive.

Active agents are attached to the present carrier molecules to create carrier/active agent (C/A) complex molecules which are introduced into a blood vessel of the subject. These C/A complex molecules pass though the endothelium of only the tumor tissue and interact with the fibrous structures of the tumor interstitium. The uptake and retention of these molecules is more than five times higher than observed for other macromolecules such as compact peptide coils or globular proteins. The penetration of the tumor interstitium by the C/A complex molecules may be enhanced by the process of reptation in which the C/A molecules are chosen, or modified to have a worm-like configuration and can “snake” around fixed obstacles in the extracellular matrix of the tumor interstitium.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawing, in which:

FIG. 1 shows a r4eaction scheme for activating a SHM (DTPA) and reacting it with a copolymer backbone.

FIG. 2 is an illustration of the functioning of a conjugated copolymer in accordance with the present disclosure in a subject.

FIG. 3 is an illustration of inter-strand and intra-strand cross-linking of polypeptides.

FIG. 4 an illustration of a highly substituted polypeptide according to the present disclosure.

### DETAILED DESCRIPTION OF THE INVENTION

The present methods employs random copolymers to reduce the degree of conjugation of substituted polypeptides required to attain a carrier molecule possessing a worm-like configuration compared to prior art molecules. The present methods and materials therefore maintain the effectiveness of the substituted polypeptides as contrast agents or drug delivery agents, but do so at a lower, more reliably attained degree of conjugation compared to prior art molecules.

The present carrier molecules include a random copolymer backbone that is substituted with groups which, due to their physical size, provide a physical restraint on peptide bond rotation.

The random copolymer forming the backbone of the carrier molecule contains lysine units and either glutamic acid units, aspartic acid units, or both. Glutamic and/or aspartic acid units may constitute from about 20 to about 60 percent of the copolymer. Preferably, the copolymer is a glutamic acid-lysine copolymer. The length of the polymer can range from about 35 residues to about 1500 residues or more. Particularly useful copolymers have glu:lys ratio of about 1:4 to about 6:4. A high content of lysine is believed advantageous for imaging as it allows a high loading of the copolymer with paramagnetic ions. Without wishing to be bound by any theory, it is believed that the presence of glutamic acid residues in the copolymer backbone accomplishes two things. First, it is believed that the glutamic acid residues provide a stiffer initial copolymer backbone for the synthesis of the complete construct. Second, it is believed that the presence of glutamic acid residues in the copolymer promotes extension of the final polymer through charge repulsion. Suitable copolymers can be synthesized using techniques known to those skilled in the art. Suitable copolymers are also commercially available from a variety of sources.

At least a portion of the lysine groups of the copolymer have a steric hindrance molecule (“SHM”) attached thereto. The SHM is any molecule that by its physical size enforces a elongated conformation by providing steric hindrance between neighboring steric hindrance molecules. Preferably the SHM is neutral in charge or presents negative charges in an aqueous environment along the polymer chain to assist in keeping the polymer backbone straight through coulombic repulsion.

In particularly useful embodiments, the SHM contains or chelates an image producing entity. Suitable image producing entities include paramagnetic entities and entities which undergo nuclear reaction to emit a particle, such as, for example, an alpha particle, a gamma particle, a beta particle, or a positron. Such imaging entities are known to those skilled in the art. Gamma emitters include, for example, ¹¹¹In and ¹⁵³Gd. Positron emitters include, for example, ⁸⁹Zr, which may be employed in positron emission tomography (PET) imaging.

Particularly preferred steric hindrance molecules are molecules that chelate with paramagnetic entities. As those skilled in the art will appreciate, paramagnetic entities include certain transition metals and lanthanide ions. Any molecule known to complex with paramagnetic entities and which is of sufficient size to provide steric hindrance against polymer bending can be used as the SHM. Preferably, the group present on the polymer backbone that is derived from the SHM exhibits a net negative charge in an aqueous environment. Suitable lanthanide ion chelating molecules include, but are not limited to diethylenetriaminepentaacetic acid (DTPA), 1,4,7,10-tetraazacyclododecane-1,4,7,10-tetraacetic acid (DOTA), 1,4,7,10-tetraazacyclododecane-1,4,7,10-tetrakis(2-propionic acid) (DOTMA), 1,4,8,11-tetraazacyclotetradecane-1,4,8,11-tetraacetic acid (TETA), 1,4,7,10-tetraazacyclododecane-1,4,7,10-tetrakis[3-(4-carboxyl)-butanoic acid], 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl-1,4,7,10-tetraazacyclododecane-1,4,7,10-tetraacetic acid (p-SCN-Bz-DOTA). Ligands useful for chelating for other ions (such as, for example, Fe(III), Mn(II), Cu(II), etc.) include bis(thiosemicarbazone) and derivatives, porphyrins and derivatives, 2,3-Bis(2-thioacetamido)propionates and derivatives, N,N′-bis(mercaptoacetyl)-2,3-diaminopropanoate, and bis(aminoethanethiol) and derivatives.

Typically, to attach the SHM to the copolymer backbone, an activating group is provided on the SHM. The activating group present on the SHM can be any group which will react with the copolymer. Suitable groups include, but are not limited to mixed carbonate carbonic anhydride groups, amine groups, succinimidyl groups and dicyclohexylcarbodiimide (DCC) groups. Those skilled in the art will readily envision reaction schemes for attaching an activating group to any given SHM.

In particularly preferred methods, a substantially mono-activated steric hindrance molecule (“SHM”) is provided. The term “activated” means that a functional group is provided on the SHM which permits covalent bonding of the molecule to the copolymer chain. By the term “substantially mono-activated” it is meant that about 90% or more of the steric hindrance molecules contain only a single activated site.

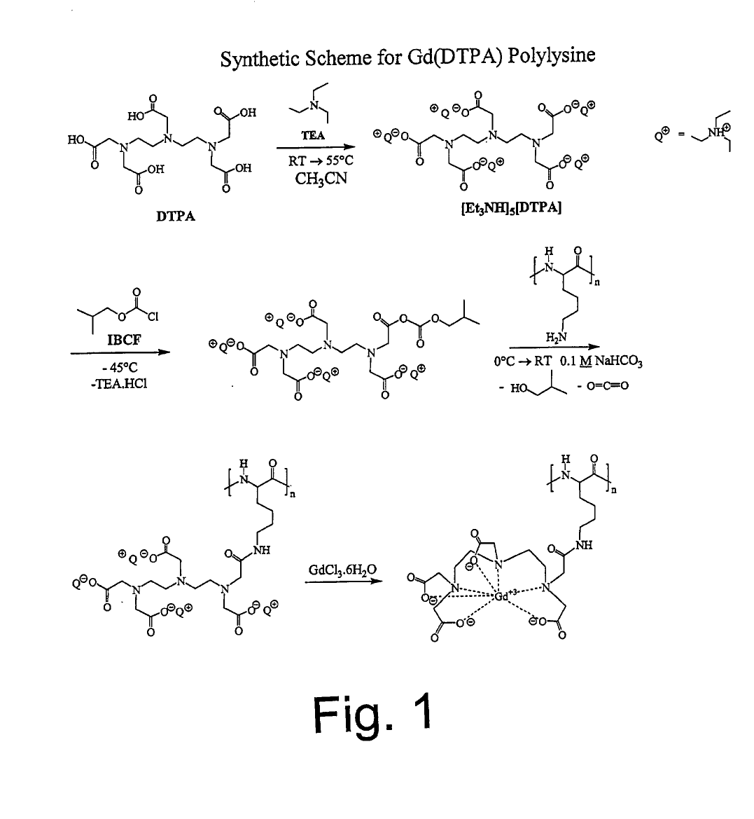

In one embodiment, the SHM is DTPA and the activating groups are mixed carbonate carbonic anhydride groups. A typical reaction scheme for activating DTPA and reacting it with a polypeptide backbone is shown in FIG. 1. As seen therein, a monoanhydride-DTPA is first prepared. Specifically, a flask is charged with acetonitrile and DTPA. Triethylamine is then added via syringe. The solution is warmed to 60° C. under a nitrogen atmosphere. The mixture is stirred until homogeneous. The clear solution is then cooled to −45° C. under nitrogen atmosphere and isobutyl chloroformate is slowly added to result in the mono-anhydride of DTPA. As those skilled in the art will appreciate, DTPA has five acid groups available for conversion to anhydride. However, since substantially mono-activated DTPA is desired, only one of these acid sites should be converted to anhydride. It has unexpectedly been found that the slow addition of the chloroformate while cooling below −40° C. accomplishes this result, i.e., that about 90% or more of the DTPA is a monoanhydride of DTPA.

The substantially mono-activated SHM is then reacted with the lysine-containing polymer.

The precise conditions for reacting the copolymer with the substantially mono-activated SHM will depend upon a number of factors including the particular copolymer chosen and the specific SHM used. Those skilled in the art will readily envision reaction schemes for any given pair of materials to produce the desired copolymer-SHM conjugates.

In a particularly useful embodiment, for example, the monoanhydride-DTPA described above is simply added dropwise to an aqueous solution of poly(lysine-co-glutamic acid) under ambient atmospheric conditions.

The resulting copolymer-SHM product is then purified. During purification, the copolymer-SHM product is separated from the volatile solvents and other impurities. Any known techniques can be used to purify the copolymer-SHM product.

In a particularly useful embodiment, a purification scheme is employed which does not result in complete drying of the copolymer-SHM product. Excessive dryness is believed to affect the configuration of the copolymer-SHM product and interferes with the determination of degree of conjugation.

A preferred purification scheme involves first exposing the reaction mixture to reduced pressure to remove impurities that are more volatile than water. Care should be taken not to remove all water from the reaction mixture during this step. The next step in this preferred purification scheme is to centrifuge the remaining reaction mixture. Soluble impurities remain in the supernatant fluid. The retentate from the centrifuge step is resuspended and subjected to dialysis. Optionally, ultrafiltration is performed on the dialyzed copolymer. Techniques for these processes are within the purview of those skilled in the art.

The resulting product can then be characterized using any technique known to hose skilled in the art, such as, for example, high performance liquid chromatography (HPLC).

In certain embodiments where the conjugated copolymers are to be used as imaging agents, an image producing entity is incorporated into the conjugated polymer. Thus, for example, to achieve a MR active agent, a paramagnetic ion can be incorporated into the copolymer-SHM product. By way of example, gadolinium can be loaded into chelating DTPA groups by dropwise addition of a gadolinium salt (e.g., GdCl₃ or gadolinium citrate in 0.1 M HCl (50 mM in Gd)) into a solution (15 mM NaHCO₃) containing the copolymer-SHM product. The dropwise addition of Gd continues until a slight indication of free Gd (not chelated by available DTPA groups) is noted (small aliquots of polymer solution added to 10 .mu.M of arzenzo III in acetate buffer—free Gd turns the dye solution blue). The Gd-loaded highly conjugated copolymer is then ready for introduction into a blood vessel of the subject.

In certain embodiments, the conjugated polymer can be used for drug delivery. It is contemplated, for example, that the SHM can itself be a therapeutic agent. It is also contemplated that a therapeutic agent can be attached at a few sites along the substituted copolymer chain. By way of example, chemotherapeutic agents (such as, for example, doxorubicin or methotrexate) which have been shown to have activity against tumors can be attached to the conjugated copolymer. Even though specific chemotherapy drugs, doxorubicin and methotrexate, are mentioned here, any known chemotherapy drugs capable of being attached to the specific polypeptide being used may be employed. Also, plant and bacterial toxins such as ricin and abrin and the like may be used. For therapy, one could alternatively use a radiotherapeutic agent such as ⁹⁰Y or ²¹¹At.

The therapeutic entity can be attached to the conjugated copolymer using techniques known to those skilled in the art. It is also contemplated that therapeutic agents can be used in combination with other types of active agents incorporated into the conjugated copolymer. For example, the copolymer backbone can be highly conjugated with a non-therapeutic SHM which chelates an image producing entity and a therapeutic agent can appear at only a few sites along the backbone. As another example, the copolymer backbone can be highly conjugated with a non-therapeutic SHM, and a therapeutic agent can be bound to the SHM, rather than being bound directly to the copolymer backbone.

In other embodiments, the conjugated polymer can be used for targeting specific tissue. It is contemplated, for example, that the SHM can itself be a targeting agent. It is also contemplated that a targeting agent can be attached at a few sites along the substituted copolymer chain. The targeting agent can be attached to the conjugated copolymer using techniques known to those skilled in the art. It is also contemplated that targeting agents can be used in combination with other types of active agents incorporated into the conjugated copolymer. For example, the copolymer backbone can be highly conjugated with a non-targeting SHM which chelates an image producing entity and a targeting agent can appear at only a few sites along the backbone. As another example, the copolymer backbone can be highly conjugated with a non-targeting SHM, and a targeting agent can be bound to the SHM, rather than being bound directly to the copolymer backbone.

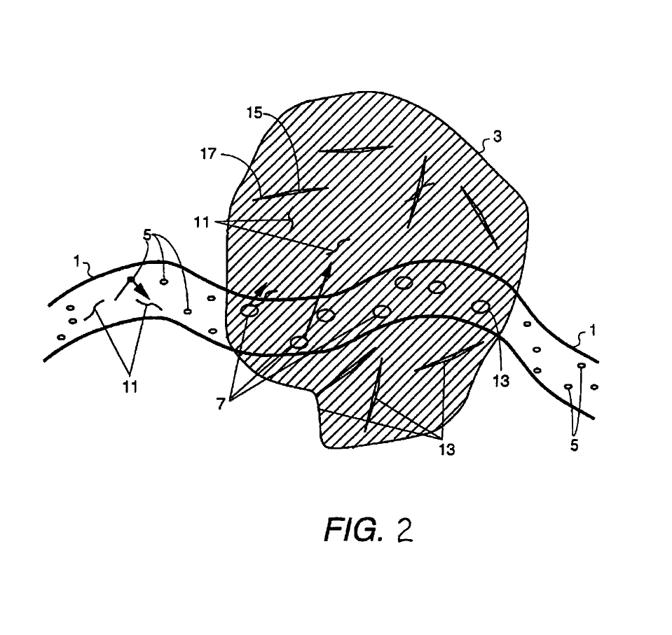

In FIG. 2 a blood vessel 1 is shown passing from normal tissue into tumor tissue 3. Pores 5 of blood vessels in the normal tissue are small and carrier/active agent (C/A) complex molecules 11 being a polypeptide carrier molecule attached to an active agent molecule, are contained in the vessels. The active agent molecules may be known image contrast enhancing agents, drugs, toxins, or other molecules which is intended to be targeted to the tumor tissue. Inside of tumor tissue 3, pores 7 are much larger than that of pores 5 in normal tissue. C/A complex molecule 13 is shown passing through pore 9, into the interstitial space of tumor 3.

Alternatively, the pores may not be simple channels but may be backed by a fibrous network of the extracellular matrix of the endothelium. A process called reptation allows elongated worm-like molecules to wiggle around obstacles, and to pass through restricted openings, that globular or coiled molecules would be unable to pass through. Experimental results suggest that a large fraction of tumor channels may in fact be restricted channels of this type rather than simple openings in the endothelium.

Stroma 17 is abundant in the interstitial space of tumor 3. C/A complex molecule 15, having the proper confirmation, size, and charge, is shown tangling with stroma 17 become entrapped in the interstitial space of tumor 3.

The present C/A complex molecules preferably have a cross sectional diameter which is larger than that of the pores of normal endothelium such that they are contained within the blood vessels in normal tissue but have a cross sectional diameter smaller than that of the pores of the vessels in tumor tissue such that they may readily pass out of the pores and into the interstitial space. Complex molecules having a diameter of approximate 20-50 Angstroms (Å) generally pass through pore structures of the tumor tissue, but not that of normal tissue.

In order to be effective at concentrating within a tumor, the C/A complex molecules also advantageously can have a length long enough to increase the time in which they circulate in the blood, but small enough to be taken up in the tumor interstitium. Once in the tumor interstitium, longer molecules tend to remain there, possibly by becoming entangled in the stroma in the interstitial space.

Concentration of the C/A molecules into tumor tissue is the product of two processes which depend upon chain length.

1. Uptake into tumor tissue by reptation, is a first process in which uptake becomes less effective as the peptide chain increases in length. Even though reptation can allow passage through obstructions and pores, the longer the molecule the more it will be retarded in its passage into tumor interstitium. This process is well known and gives rise to the separation of DNA molecules or denatured proteins in gel electrophoresis.

2. The second process involves clearance of the C/A complex molecules from the blood circulation performed by glomerular filtration of the kidneys. Clearance is rapid for short molecules, resulting in a short plasma lifetime. Plasma lifetime increases rapidly as the peptides increase in length but a plateau is reached for a molecular length of about 500 residues and little further change in lifetime occurs.

An elongated, worm-like conformation of a macromolecule results in greater uptake than other conformations, such as folded, or globular conformations. Conformation may be measured by a persistence length of the molecule. This may be determined by light scattering.

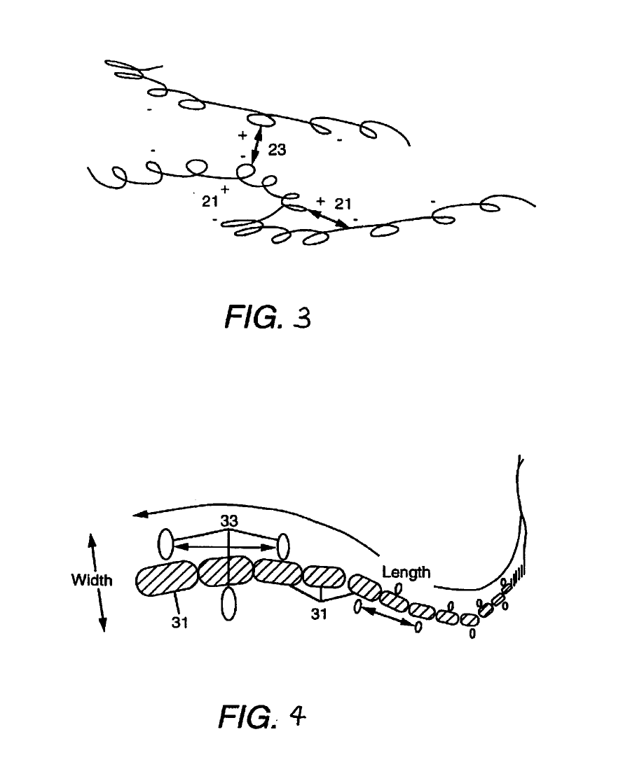

Conformation is a result of intra-chain charge interaction, and rigidity of the molecule. C/A carrier molecules are selected to be polypeptides. However, many polypeptides tend to fold into tight random coils due to the relatively free rotation around each peptide bond. Also, if each polypeptide is composed of opposite charge amino acids, then intra-chain charge interaction as shown by bond 21 in FIG. 3. Inter-chain charge interaction between chains may also occur as shown by bond 23 of FIG. 3. If there is significant intra-chain charge interactions, the C/A complex molecules may assume a globular, or folded, conformation.

The conformation attained by the present random copolymer carrier molecules is that of a worm-like shape being essentially a stretched out, extended chain with little folding. A measure of the “straightness” of a molecule is a persistence length. Persistence length is related to a radius of gyration, measured by light scattering experiments. A folded polypeptide such as poly-L-lysine (PLL) with little or no substitution, has a low persistence length of about 10 Angstroms (Å), and is not suitable for targeting tumor tissue. Therefore, the present random copolymer-based C/A complex molecules preferably have a persistence lengths of 100-600 Å and thus concentrate much more readily in tumor tissue than C/A complex molecules of PLL. In order to produce a carrier molecule and active agent complex having a proper persistence length, the random copolymer starting material substantially eliminates or reduces intra-chain ionic bonding.

It is sometimes difficult to measure the persistence length of certain molecules by light scattering to determine their conformation because of the effects of contaminant particles in the test solutions. However, it was found that by measuring the magnetic resonance (MR) T₁ relaxation of a paramagnetic entity attached to the carrier, one could infer the conformation of the molecules of interest. This is performed by attaching paramagnetic ions, such as gadolinium, to the chelators along the polymer chain

When the carrier molecule is in an elongated conformation, the chelator/MR active entity is free to rotate about its attachment point to the main chain, allowing a long T₁ relaxation time of the surrounding water protons which are the source of the MR signal.

When the carrier molecule is in a globular or highly folded conformation, steric hindrance, and molecular crowding causes interaction with the chelator/MR active entity restricting rotation about its bond to the main chain. Thus, the chelator/MR active entity moves only with the general slow motion of the carrier molecule. This produces a short T₁ relaxation time.

A high relaxivity is associated with a molecule which folds upon itself into a globular conformation, such as albumen, at about 15 sec.⁻¹ milliMolar⁻¹ (sec.⁻¹ mM⁻¹). A low relaxivity is associated with an elongated molecule such as highly substituted Gd-DTPA PLL.sup.h in which the Gd can rotate rapidly, having a relaxivity of about 8 sec.⁻¹ mM⁻¹. The optimum conformation of the present invention is associated with a relaxivity of 7-8 sec.⁻¹ mM⁻¹. When the relaxivity of a peptide agent was high, the uptake coefficient of such an agent was invariably low, evidently due to the absence of the reptation mechanism.

Since many in-vivo chemical entities have a negative charge, molecules introduced into the subject can advantageously have a net negative charge to reduce agglutination and to allow for stable long circulation in the blood plasma. It is known that negatively charged dextran molecules undergo glomerular filtration at a much slower rate than equivalent dextran molecules of positive charge or neutral charge.

The high net negative charge is also desirable since it also assists in the C/A complex molecules to retaining their elongated, worm-like conformation.

In FIG. 4 a copolymer carrier having a plurality of side chains substituting the hydrogen atoms is shown. The copolymer is comprised of a plurality of amino acids 31, each linked end to end through a polypeptide bond. A plurality of side residues 33 are attached which cause steric hindrances and repulsion to straighten the copolymer chain.

FIG. 4 also shows that the length of the copolymer should be significantly longer than its diameter by approximately 5 to 500 times. This causes the copolymer and any attached chemical entities to pass through pores in tumor tissue and become trapped the tumor interstitium as discussed above.

Since many in vivo molecules tend to have a negative charge, it is advantageous for the C/A complex molecules to also have a net negative charge in order to avoid agglutination with blood plasma proteins. Positively charged molecules are also known to stick to cell surfaces (which are generally negatively charged).

In order to perform one preferred method of using the present compositions, a subject is first imaged and then a copolymeric contrast agent in accordance with this disclosure is introduced into the subject by injecting the contrast agent intravenously. The dose of the polymeric contrast agent can be in the range of about 0.01 mmoles Gd/Kg to about 0.1 mmoles Gd/Kg. The subject is then imaged at one or more pre-selected tissue sites. The subject is imaged, preferably beginning immediately after injection and at certain timed intervals. Preferably, the timed intervals are shortly after injection (within 10 minutes) and up to 1 hour post injection. An image at 24 hours may also be acquired.

### EXAMPLE 1

A flask is charged with acetonitrile and DTPA. Triethylamine is then added via syringe. The solution is warmed to 60° C. under a nitrogen atmosphere. The mixture is stirred until homogeneous. The clear solution is then cooled to −28° C. under nitrogen atmosphere and isobutyl chloroformate is slowly added to result in the anhydride of DTPA. Anhydride of DTPA was reacted for 12 hours with a glutamic acid-lysine copolymer (glu:lys=6:4 with a polymerization number of 140 obtained from Sigma Chemicals) at a DTPA to lysine ration of 7:1. The product was subjected to rotovap for 20 minutes at 50° C. and then diafiltration purification through 30,000 Mw cutoff membrane filters.

Yield was 22%. The free lysine fraction uncoupled to total lysine available was 20% and the equivaqlent to lysine/total number of residues was determined with a TNBS assay (as described in Fields, Methods in Enzymology, 25:464-468 (1972)) to be 8%. R1, the T1 relaxivity at 23° C. was 8.5 mMsec. Although lysine conjugation was poor (20%), the effective conjugation due to dilution of positive lysine charges by glutamic acid residues is a very good 8%.

Imaging was done on a rat tumor model. Specifically, rat mammary adenocarcinoma cells (ATCC 13762, Mat B cells) were implanted in Fisher 344 female rats (106 cells in 0.5 ml phosphate buffered saline). After 7-9 days, tumors were about 10 mm in diameter and the imaging was done at that point. Imaging was performed using a GE CSI scanner at 2T with a 33 cm bore. A birdcage quadrature coil was used for transmission and receiving. T1 weighted images were obtained (TR 250 ms, TE 18 MS, NEX 16). Rats were imaged prior to injection of contrast agent. Contrast agent was injected by tail vain at a dose of 0.025 mmole Gd/kg. The rats were then imaged immediately after injection and then at 24 hours. Imaging efficacy was 67% tumor enhancement compared to 15% for globular (albumin (Gd)DTPA) or coiled (25-35% free lysine content) agents at the same dose.

### EXAMPLE 2

The synthesis of Example 1 was followed except that a 1:4 glu-lys copolymer having a polymerization number of 1013 obtained from Sigma Chemicals was used as the copolymer. Yield was 30%. The free lysine fraction uncoupled to total lysine available was 12% and the equivaqlent to lysine/total number of residues was 10%. R1, the T1 relaxivity at 23° C. was 7.9 mMsec., a very good value consistent with an extended copolymer. After 24 hours, tumor signal enhancement was 30%. Again, globular or coiled polymers gave only a 10 to 15% signal enhancement.

It may be that in some applications, long blood circulation times would be undesirable. The present methods/materials provide the ability to reliably make short polymers of the desired worm-like conformation which allows the possible tailoring of blood circulation time to certain target levels. Blood circulation time is directly dependent on polymer chain length. Although tumor enhancement is diminished for short polymers according to theoretical expectations, the response is fast (less than 1 hour) and the clearance from the blood circulation is rapid, both of which may be desirable in certain clinical screening procedures. In any case, for the short polymers prepared in accordance with certain embodiments described herein, tumor enhancement is larger by more than a factor of two compared to the responses obtained for the currently FDA approved contrast agent, Gd-DTPA. However, the present short random copolymers agent does not rapidly wash out of tumors as does Gd-DTPA. Therefore, clinical screening procedures would be much simplified over the small molecular weight agent now in use.

In the case of short backbone prior art polymer materials, difficulty achieving proper conformation is typically encountered following standard synthesis schemes. Possibly because of end to end interactions, an extended conformation is not achieved, as evidenced by high proton relaxivity and low tumor enhancement efficacy. However, following the same standard synthesis procedures starting with a random copolymer of only 140 residues, proton relaxivity was low indicating an extended form for the product. The tumor enhancement in a rat tumor model was 160% for a standard dose of 0.1 mmol Gd/kG compared to less than 5% for a coiled agent of Gd-DTPA-polylysine of 90 residue chain length, or compared to 70% for Gd-DTPA, a small molecular weight contrast agent. Furthermore, this level of enhancement for a chain length of 140 residues is exactly what would be predicted from a reptation process given the observed enhancement at a chain length of 476 residues for an extended homopolymer.

In addition to tumor visualization, the shorter random copolymer agents in accordance with the present disclosure can advantageously be used in other applications. With relatively good blood clearance properties, the present intravascular polymeric agents may be useful for angiography. They also do not appear to accumulate in other organs such as muscle, kidney or liver. Therefore, the present agents may be preferred for drug delivery/imaginig over others that are based on globular proteins or coiled homopolymers, which tend to show accumulation in liver and kidneys of animal models.

While specific embodiments of the invention have been illustrated and described herein, it is realized that modifications and changes will occur to those skilled in the art. It is therefore to be understood that the appended claims are intended to cover all such modifications and changes as fall within the true spirit and scope of the invention.

What we claim is:

1. A molecule comprising a polypeptide containing lysine residues and one or more types of amino acid residues selected from the group consisting of gluatmic acid residues and aspartic acid residues, less than 90% of the lysine residues being substituted with a group derived from a steric hindrance molecule, the substituted polypeptide having a conformation with a length that is 5 to 500 times its average diameter.

2. A molecule as in claim 1 wherein the group derived from a steric hindrance molecule is capable of chelating an image producing entity.

3. A molecule as in claim 1 wherein the group derived from a steric hindrance molecule is capable of chelating a paramagnetic entity.

4. A molecule as in claim 1 wherein the steric hindrance molecule is selected from the group consisting of diethylenetriaminepentaacetic acid (DTPA), 1,4,7,10-tetraazacyclododecane-1,4,7,10-tetraacetic acid (DOTA), 1,4,7,10-tetraazacyclododecane-1,4,7,10-tetrakis(2-propionic acid) (DOTMA), 1,4,8,11-tetraazacyclotetradecane-1,4,8,11-tetraacetic acid (TETA), 1,4,7,10-tetraazacyclododecane-1,4,7,10-tetrakis[3-(4-carboxyl)-butanoic acid], 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and p-isothiocyanatobenzyl-1,4,7,10-tetraazacyclododecane-1,4,7,10-tetraacetic acid (p-SCN-Bz-DOTA), bis(thiosemicarbazone), bis(thiosemicarbazone) derivatives, porphyrins, porphyrin derivatives, 2,3-bis(2-thioacetamido)propionates, 2,3-bis(2-thioacetamido)propionate derivatives, N,N′-bis(mercaptoacetyl)-2,3-diaminopropanoate, bis(aminoethanethiol) and derivatives of bis(aminoethanethiol).

5. A molecule as in claim 1 wherein the polypeptide is a random copolymer of lysine and glutamic acid.

6. A molecule as in claim 1 wherein the polypeptide contains lysine and glutamic acid residues in a ratio ranging from 1:4 to 6:4.

7. A molecule as in claim 1 wherein the polypeptide is a random copolymer containing 20 to 60 percent glutamic acid residues, the balance of the polypeptide being lysine residues.

8. A molecule as in claim 1 wherein the polypeptide comprises from 35 to 1500 amino acid residues.

9. A molecule as in claim 1 wherein the substituted polypeptide has an average diameter of 20 to 50 angstroms.

10. A molecule as in claim 1 wherein the polypeptide is a random copolymer of lysine and glutamic acid and the steric hindrance molecule is diethylenetriaminepentaacetic acid.

11. A molecule as in claim 1 further comprising an image producing entitiy.

12. A molecule as in claim 11 wherein the image producing entitiy is a paramagnetic entity.

13. A molecule as in claim 11 wherein the image producing entitiy is a lanthanide ion.

14. A molecule as in claim 11 wherein the image producing entitiy is gadolinium.

15. A molecule as in claim 1 further comprising a theraprutic agent.

16. A molecule as in claim 1 further comprising a targeting agent.

17. A molecule comprising a random copolymer of lysine and glutamic acid wherein less than 90% of the lysine residues are substituted with groups derived from diethylenetriaminepentaacetic acid, the substituted copolymer having a conformation with a length that is 5 to 500 times its average diameter, at least a portion of the groups derived from diethylenetriaminepentaacetic acid having a gadolinium ion associated therewith.

18. A method comprising administering a compound in accordance with claim 11 to a subject and imaging the subject.

19. A method as in claim 18 wherein the compound is a random copolymer of lysine and glutamic acid substituted with groups derived from diethylenetriaminepentaacetic acid, at least a portion of the groups derived from diethylenetriaminepentaacetic acid having a gadolinium ion associated therewith.

20. A method as in claim 19 wherein the compound is administered at a dose in the range of 0.01 mmoles Gd/Kg to about 0.1 mmoles Gd/Kg.
